(19) United States
(12) Patent Application Publication
(10) Pub. No.: US 2015/0005697 A1
(43) Pub. Date: Jan. 1, 2015

(54) APPLICATOR

(71) Applicant: TERUMO KABUSHIKI KAISHA, Shibuya-ku (JP)

(72) Inventors: Teruyuki YATABE, Ashigarakami-gun (JP); Kenichi Shimura, Ashigarakami-gun (JP)

(73) Assignee: TERUMO KABUSHIKI KAISHA, Tokyo (JP)

(21) Appl. No.: 14/484,986

(22) Filed: Sep. 12, 2014

Related U.S. Application Data
(63) Continuation of application No. PCT/JP2013/055984, filed on Mar. 5, 2013.

(30) Foreign Application Priority Data
Mar. 15, 2012 (JP) 2012-058266

(51) Int. Cl. A61M 25/00; A61M 25/06; A61M 11/00

(52) U.S. Cl. CPC A61M 25/0023; A61M 11/007; A61M 25/0662; A61M 2025/0681
USPC 604/24

(57) ABSTRACT

An applicator is inserted into a living body and applies mixed solution to a region in the living body. The applicator includes a nozzle including an elongated nozzle main body to which gas and a plurality of kinds of liquids are supplied and a nozzle head at a distal end of the nozzle main body and configured to jet A mixed solution of the gas and the liquids supplied to the nozzle main body, and a sheath in which the nozzle main body is fitted for relative movement along a longitudinal axial direction of the nozzle main body. At least two crushed portions are provided contiguously to each other at a proximal end portion of the nozzle main body at the opposite side to the nozzle head such that deformation directions thereof with respect to an axial line of the nozzle main body are different from each other.

### CROSS-REFERENCES TO RELATED APPLICATIONS

This application is a continuation of International Application No. PCT/JP2013/055984 filed on Mar. 5, 2013, and claims priority to Japanese Application No. 2012-058266 filed on Mar. 15, 2012, the entire content of both of which is incorporated herein by reference.

### TECHNICAL FIELD

The present invention generally relates to an applicator for applying an anti-adhesion material, a biological tissue adhesive or the like to an affected area or the like, and particularly to an applicator suitable for use in a laparoscopic operation.

### BACKGROUND DISCUSSION

Conventionally, a method is known wherein two or more kinds of liquids are mixed and injected to an affected area or the like to form an anti-adhesion material, a biological tissue adhesive or the like, and an applicator for the method has been developed.

A conventional applicator is configured such that components which solidify when they are mixed, for example, solution containing thrombin and solution containing fibrinogen are fed in a separated state from each other to a location in the proximity of an affected area and are applied while being mixed at the affected area.

As such an applicator as just described, an applicator is available which includes two syringes individually containing different kinds of liquids and a nozzle which mixes and jets the liquids from the syringes. An example is disclosed in U.S. Application Publication No. 2010/0331766.

The applicator described in U.S. Application Publication No. 2010/0331766 includes a nozzle and a sheath. The nozzle includes a nozzle main body of an elongated tubular shape and a nozzle head provided at a distal end of the nozzle main body. At a distal end portion of the nozzle main body, a curved portion having flexibility and curved or bent is formed. The sheath corrects, when the nozzle main body is fitted into the sheath for movement along a longitudinal (axial) direction of the sheath and the curved portion is inserted into the sheath, the shape of the curved portion to adjust the direction of the nozzle head with respect to an axial line of the nozzle main body. A gap is formed in the longitudinal (axial) direction between the sheath and the nozzle. The gap functions as an exhaust path for exhausting, when the abdominal pressure in the abdominal cavity rises, the gas in the abdominal cavity to the outside of the body through the gap.

In U.S. Application Publication No. 2010/0331766, one flattened portion is formed at a portion at the proximal end (proximal end portion) of the nozzle main body. The flattened portion is greater than the inner diameter of the sheath in a natural state, and an outer peripheral face of the flattened portion of the nozzle main body closely contacts with the inner peripheral face of the sheath. When, in this state, the sheath is moved along a longitudinal (axial) direction thereof, the inner peripheral face of the sheath slidably moves on the outer peripheral face of the flattened portion. Thereupon, frictional resistance is generated between the inner peripheral face of the sheath and the outer peripheral face of the flattened portion. This provides sliding resistance to stop the sheath at a predetermined position of the nozzle main body, and the shape of the curved portion is regulated by the distal end of the sheath. For example, as depicted in FIG. 6, the flattened portion 112 is formed by collapsing part of the tubular nozzle main body 110 from the opposite sides in a direction perpendicular to the plane of FIG. 6 so as to contact with the inner face 114a of the sheath 114.

### SUMMARY

In the applicator described in U.S. Application Publication No. 2010/0331766, the flattened portion is formed at a portion at the proximal end (proximal end portion) of the nozzle main body to generate frictional resistance between the inner peripheral face of the sheath and the nozzle main body as described above. However, if the shape of the sheath varies by heating upon sterilization of the applicator such as upon autoclave sterilization or ethylene oxide gas sterilization or by time dependent variation or the like of the applicator, then the sliding resistance decreases, resulting in failure to stop the sheath. Consequently, there is the possibility that the curved portion at the distal end of the nozzle main body cannot be regulated to the predetermined shape, resulting in deterioration of the operability.

The applicator disclosed here is configured in a manner that reduces the likelihood of a reduction in the sliding resistance by heating upon sterilization or by time-dependent variation and regulate the shape of a curved portion at the distal end of a nozzle main body accurately by a sheath and besides has a stable operability over a long period of time.

The applicator includes a nozzle including an elongated nozzle main body to which gas and a plurality of kinds of liquids are supplied and a nozzle head provided at a distal end of the nozzle main body and configured to jet mixed solution of the gas and the plurality of kinds of liquids supplied to the nozzle main body and a sheath in which the nozzle main body is fitted for relative movement along a longitudinal (axial) direction of the nozzle main body, the applicator being inserted into a living body to apply the mixed solution to a region in the living body, at least two crushed portions being provided contiguously to each other at a proximal end portion of the nozzle main body at the opposite side to the nozzle head such that deformation directions thereof with respect to an axial line of the nozzle main body are different from each other, the at least two crushed portions individually contacting with an inner face of the sheath.

Preferably, the different deformation directions of the at least two crushed portions define an angle of 90±20 degrees therebetween. Typically, the crushed portions are flattened portions of the nozzle main body deformed by being crushed from opposite sides.

With the applicator disclosed here, a drop in the sliding resistance by heating upon sterilization or by time-dependent variation is suppressed and the shape of the curved portion at the distal end of the nozzle main body can be regulated accurately by the sheath and besides a stable operability can be obtained over a long period of time.

According to another aspect, an applicator configured to be positioned in a living body comprises: a sheath possessing a lumen, an inner face surrounding the lumen, and open distal and proximal ends communicating with the lumen of the sheath; and a nozzle including an elongated nozzle main body possessing proximal and distal ends, a spray head connectable to different kinds of liquids to be conveyed through the nozzle main body and mixed with one another, and a nozzle head. The spray head is fixed to the proximal end of the nozzle main body so that the nozzle main body and the spray head move together as a unit, with the nozzle head being fixed to the distal end of the nozzle main body so that the nozzle main body and the nozzle head move together as a unit. The nozzle head possesses a distal end at which is located an opening through which the liquids mixed together as a mixed solution are ejected together with a gas, with the nozzle main body being axially movably positioned in the lumen of the sheath. The applicator is insertable into a living body to apply the mixed solution to a region in the living body, and the nozzle main body includes an axially extending first portion at which the nozzle main body is deformed in a first deformation direction relative to a portion of the nozzle main body axially adjacent the first portion so that an outer circumferential part of the axially extending first portion of the nozzle main body contacts the inner face of the sheath while the portion of the nozzle main body axially adjacent the first portion is spaced from the inner face of the sheath. The nozzle main body includes an axially extending second portion at which the nozzle main body is deformed in a second deformation direction relative to a portion of the nozzle main body axially adjacent the second portion so that an outer circumferential part of the axially extending second portion of the nozzle main body contacts the inner face of the sheath while the portion of the nozzle main body axially adjacent the second portion is spaced from the inner face of the sheath. The first and second portions of the nozzle body are located axially contiguous to one another and at a proximal end portion of the nozzle main body, and the first and second deformation directions are different directions.

In accordance with another aspect, an applicator configured to be positioned in a living body comprises: a sheath possessing lumen, and open distal and proximal ends communicating with the lumen of the sheath, wherein the sheath also possesses an inner face surrounding the lumen; and a nozzle including an elongated nozzle main body possessing an outer surface, a spray head connectable to different kinds of liquids to be conveyed along the nozzle main body and mixed with one another, and a nozzle head. The nozzle main body possesses a distal end and a proximal end, with the spray head being fixed to the proximal end of the nozzle main body so that the nozzle main body and the spray head move together as a unit. The nozzle head is fixed to the distal end of the nozzle main body so that the nozzle main body and the nozzle head move together as a unit, with the nozzle head possessing a distal end at which is located an opening through which the liquids mixed together as a mixed solution are ejected together with a gas, the nozzle main body being axially movably positioned in the lumen of the sheath. The applicator is insertable into a living body to apply the mixed solution to a region in the living body; and the nozzle main body includes an axially extending first portion and an axially extending second portion which are contiguous to one another at a proximal end portion of the nozzle main body. One circumferential part of the axially extending first portion possesses an outer dimension in a first direction that is greater than the outer dimension of an other circumferential part of the axially extending first portion, with the outer dimension of the one circumferential part of the axially extending first portion of the nozzle main body also being greater than the outer dimension of a portion of the nozzle main body axially adjacent the axially extending first portion. One circumferential part of the axially extending second portion possesses an outer dimension in a second direction that is greater than the outer dimension of an other circumferential part of the axially extending second portion, with the outer dimension of the one circumferential part of the axially extending second portion of the nozzle main body also being greater than the outer dimension of a portion of the nozzle main body axially adjacent the axially extending second portion, and wherein the first direction is a direction that is different from the second direction. The one circumferential part of the axially extending first and second portions contacts the inner face of the sheath, and the outer surface of the portion of the nozzle main body axially adjacent the first portion is spaced from the inner face of the sheath, and the outer surface of the portion of the nozzle main body axially adjacent the second portion being spaced from the inner face of the sheath.

### BRIEF DESCRIPTION OF DRAWINGS

FIG. 1 is a schematic view depicting an applicator according to an embodiment representing one example of the applicator disclosed here.

FIG. 2 is a schematic perspective view depicting the applicator shown in FIG. 1.

FIG. 3(a) is a schematic cross-sectional view depicting a crushed portion of the applicator of the embodiment of the present invention, and FIG. 3(b) is a schematic perspective view depicting the crushed portion of the applicator of the embodiment of the present invention.

FIGS. 4(a) and 4(b) are schematic views depicting an apparatus used for a measuring method of sliding resistance.

FIGS. 5(a) and 5(b) are schematic views illustrating usage patterns of the applicator of the embodiment of the present invention, and FIG. 5(c) is a schematic view of a usage pattern of a conventional applicator.

FIG. 6 is a schematic view particularly depicting a flattened portion of U.S. Application Publication No. 2010/0331766.

### DETAILED DESCRIPTION

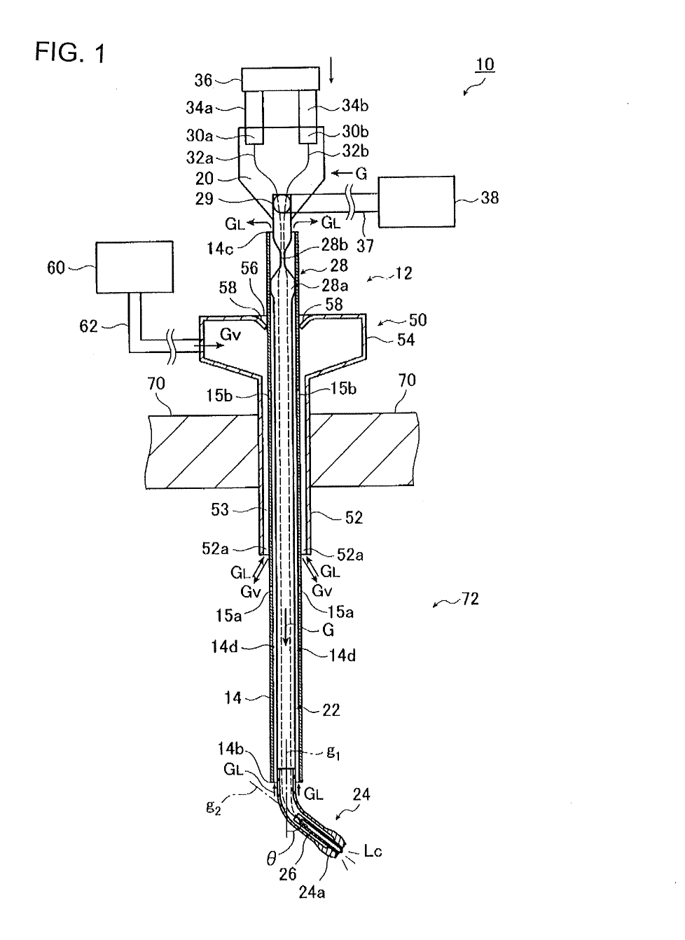

The applicator 10 depicted in FIG. 1 is an applicator configured to be inserted into an abdominal cavity 72 upon laparoscopic operation to apply anti-adhesion material or biological tissue adhesive formed by mixing two kinds of liquids having different compositions from each other to an affected area such as an organ or an abdominal wall 70. The insertion of the applicator 10 into the abdominal cavity 72 is carried out through a trocar 50 indwelled in advance in the abdominal wall 70.

First, the trocar 50 is described.

The configuration of the trocar 50 is not restricted specifically, but various known trocars which are used in laparoscopic operation can be used. For example, it is possible to use the trocar tube disclosed in FIG. 1 of Japanese Patent Laid-Open No. 2009-226189 (U.S. Application Publication No. 2010/0331766). Such a trocar 50 as just described is depicted in FIG. 1

As depicted in FIG. 1, the trocar 50 includes a hollow hub 54 communicating with a main body 52 in the form of a pipe. The hub 54 has a diameter greater than that of the main body 52 and is communicated with the main body 52.

An applicator 10 hereinafter described in detail is inserted into the main body 52. When the applicator 10 is inserted in the main body 52, a gap 53 is generated between the inner surface of the main body 52 and the outer surface of a sheath 14 of the applicator 10.

Further, the main body 52 of the trocar 50 may have a distal end opening inclined with respect to an axis of the main body 52. This makes it possible to carry out insertion of the trocar 50 into the abdominal cavity 72 readily.

The hub 54 is connected to a gas supplying unit 60 through a tube 62. The gas supplying unit or gas source 60 includes a gas tank in which sterile gas Gv is filled at a high pressure. From the gas supplying unit 60, the sterile gas Gv is supplied to the abdominal cavity 72 passing the tube 62, the inside of the hub 54 and the inside of the main body 52 in order. An on-off valve for controlling the sterile gas Gv so as to be supplied or stopped is installed in the hub 54, gas supplying unit 60 or tube 62. When sterile gas is to be supplied into the abdominal cavity 72, the valve is placed into an open state. The sterile gas Gv is, for example, air or nitrogen gas.

The hub 54 has an opening 56 formed at the side that is opposite to the side at which the main body 52 is provided. A valve 58 covers the opening 56. The valve 58 is, for example, a duckbill valve. The applicator 10 or the like is inserted into the opening 56 of the trocar 50, and the applicator 10 is inserted into the abdominal cavity 72.

The valve 58 closes the opening 56 in a state in which the applicator 10 is not inserted in (positioned in) the opening 56, but is opened when the applicator 10 is inserted through the sheath 14 of the applicator 10 and the opening 56 is placed in a sealed state against the sheath. In a state in which the applicator 10 is not inserted, and also in a state in which the applicator 10 is inserted, the sterile gas Gv is prevented from flowing out of the opening 56 and the sterile gas Gv is supplied into the abdominal cavity 72 with certainty by the valve 58.

The main body 52 and the hub 54 may be formed integrally with each other or may be formed as separate members which are connected and fixed to each other.

A pressure sensor for measuring the pressure in the abdominal cavity 72 is connected to the trocar 50. A control unit is further provided which causes the sterile gas Gv to be supplied from the gas supplying unit 60 into the abdominal cavity 72 based on the pressure obtained by the pressure sensor. Thus, under the control of the control unit, the sterile gas Gv is supplied from the gas supplying unit 60 into the abdominal cavity 72 until the intraperitoneal pressure (abdominal pressure) in the abdominal cavity 72 is raised approximately 8 to 12 mmHg above the atmospheric pressure to inflate the abdominal cavity 72. On the other hand, if the pressure in the abdominal cavity 72 drops due to leakage of the gas G_{L} in the abdominal cavity 72, then the sterile gas Gv is supplied from the gas supplying unit 60 into the abdominal cavity 72 under the control of the control unit so that the intraperitoneal pressure is kept at the pressure higher by approximately 8 to 12 mmHg than the atmospheric pressure. The abdominal cavity 72 is controlled to a magnitude sufficient to carry out laparoscopic operation using the trocar 50 in this manner. Now, the applicator 10 is described.

The applicator 10 includes a nozzle 12 and a sheath 14 into which the nozzle 12 is inserted or in which the nozzle 12 is positioned. The nozzle 12 includes a spray head 20, a nozzle main body 22 in the form of a pipe, and a nozzle head 24. In the illustrated embodiment, the nozzle head 24 is fixed to the distal end of the nozzle main body 22 so that the nozzle main body 22 and the nozzle head 24 move together as a unit, and the spray head 20 is fixed to the proximal end of the nozzle main body so that the nozzle main body and the spray head move together as a unit. The nozzle head 24 is curved in a state in which no external force is applied to the nozzle head 24.

The spray head 20 has a substantially pentagonal shape as viewed in plan and the nozzle main body 22 is connected to an apex angle portion of the spray head 20. On the nozzle main body 22, the nozzle head 24 is provided at an end portion (hereinafter referred to also as distal end portion) at the side (end) that is opposite to the side (end) at which the spray head 20 is provided. The nozzle main body 22 and the nozzle head 24 are fitted for relative movement along a longitudinal (axial) direction of the nozzle main body in the sheath 14.

By way of example, the length of the nozzle main body 22 is 30 cm; the outer diameter of the nozzle main body 22 is 3.7 mm; and the inner diameter of the sheath 14 is 4.5 mm. Therefore, a gap 14d of 0.4 mm is defined between an inner surface 14a of the sheath 14 and an outer surface of the nozzle main body 22. The gap 14d functions as an exhaust path along which the gas G_{L} in the abdominal cavity 72 is exhausted to the outside of the body when the pressure in the abdominal cavity 72 rises. The gas G_{L} in the abdominal cavity 72 passes through the gap 14d from a distal end portion 14b of the sheath 14 and is exhausted to the outside of the body past a rear end portion 14c of the sheath 14. In this case, the distal end portion 14b of the sheath 14 functions as an intake port for gas, and the rear end portion 14c of the sheath 14 functions as a gas leak exit (gas vent).

The sheath 14 is an elongated pipe or tubular body open at opposite ends, and part of the nozzle head 24 and the nozzle main body 22 are fitted inside the sheath 14. The sheath 14 possesses a lumen extending throughout the length of the sheath, and open distal and proximal ends communicating with the lumen. In the present embodiment, the sheath 14 extends from a location on the distal end side with respect to the curved portion of the nozzle head 24 to a location in the proximity of the connection location of the nozzle main body 22 to the spray head 20. Further, the sheath 14 is movable along the longitudinal (axial) direction of the nozzle main body 22 relative to the nozzle main body 22 and the nozzle head 24.

Further, the sheath 14 has a function as a shape regulation member for regulating the shape of the curved portion of the nozzle main body 22 as hereinafter described.

The sheath 14 has a plurality of side holes formed on a circumference of the sheath 14 at a plurality of positions in the longitudinal (axial) direction, and the side holes are spaced by equal distances from each other along the longitudinal direction of the sheath. More particularly, two circumferentially spaced apart side holes 15a are formed at positions at the distal end portion 14b side of the sheath 14 along the longitudinal direction, and two other side holes 15b are formed at positions at the rear end portion 14c side of the sheath 14. Thus, the two side holes 15a, 15a are located at the same axial or longitudinal position along the length of the sheath 14, and the other two side holes 15b, 15b are located at the same axial or longitudinal position along the length of the sheath 14.

Here, the side holes 15a and 15b are formed such that the distance between the distal end 14b of the sheath 14 and the position of the side holes 15a, the distance between the positions of the side holes 15a and the side holes 15b, and the distance between the position of the side holes 15b and the rear end 14c are equal to each other along the longitudinal (axial) direction of the sheath 14.

At the positions in the longitudinal (axial) direction, the two side holes 15a and the two side walls 15b are formed at an equal distance in a circumferential direction of the sheath 14. In other words, the two side holes 15a are positioned in opposing relation (diametrically opposite positions) and the two side holes 15b are positioned in opposing relation (diametrically opposite positions).

The side holes 15a and 15b extend through the sheath 14 and communicate with the gap 14d, and function as inlets through which the gas G_{L} in the abdominal cavity 72 flows into the gap 14d, namely, as intake ports through which the gas G_{L} in the abdominal cavity 72 is taken in.

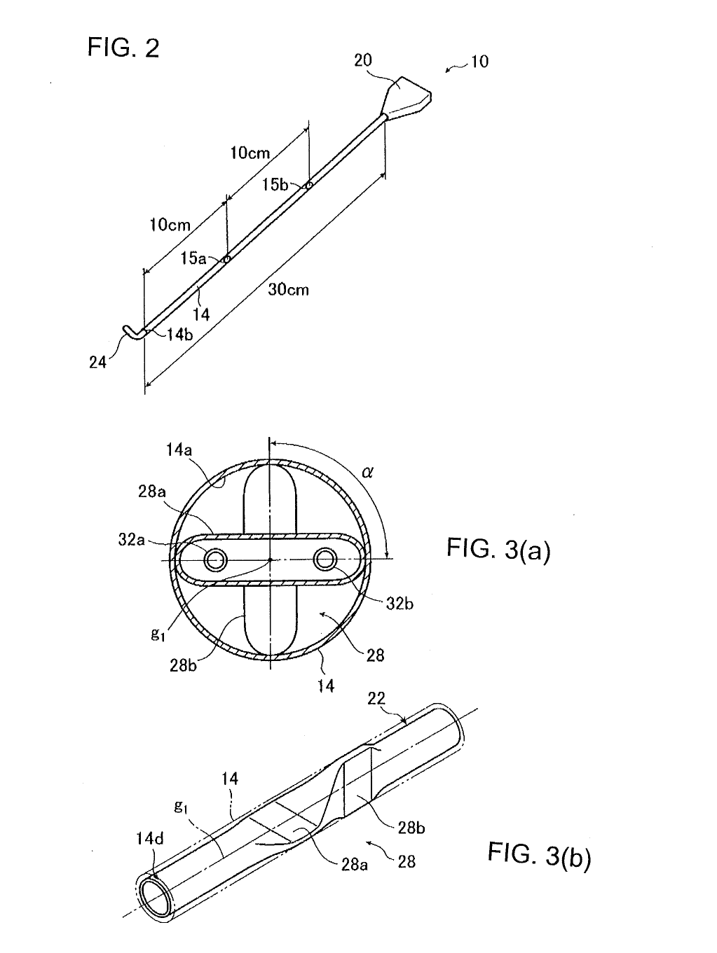

If, for example, the total length of the sheath 14 is 30 cm as depicted in FIG. 2, then the side holes 15a are positioned at an interval of 10 cm from the distal end portion 14b along the longitudinal (axial) direction of the sheath 14 and the side holes 15b are formed at a position of 20 cm from the distal end portion 14b along the longitudinal (axial) direction of the sheath 14. The interval at which the side holes are formed is not limited to 10 cm but may be 5 cm.

The gas leak amount varies depending upon the distance between the side holes and the rear end portion 14c which functions as a gas leak exit. As the distance to the rear end portion 14c decreases, the gas leak amount increases. Therefore, the gas leak amount is greater from the side holes 15b than from the side holes 15a.

By forming the plurality of side holes 15a and the plurality of side holes 15b at the plurality of positions of the sheath 14 along the longitudinal (axial) direction, in addition to a route along which the gas G_{L} in the abdominal cavity 72 passes from the distal end portion 14b of the sheath 14 through the gap 14d and is exhausted to the outside of the body through the rear end portion 14c (gas leak exit) of the sheath 14, another route is formed if the side holes exist in the abdominal cavity 72. In particular, along the latter route, the gas G_{L} in the abdominal cavity 72 passes through the side holes and are exhausted to the outside of the body past the gap 14d and the rear end portion 14c.

On the other hand, when the side holes are positioned in the trocar 50, a further route is formed. In particular, along the further route, the gas G_{L} in the abdominal cavity 72 enters the main body 52 of the trocar 50 through a distal end portion 52a, passes the gap 53 between the main body 52 and the sheath 14 and further passes the side holes 15b and the gap 14d of the sheath 14 and is then exhausted to the outside of the body from the rear end portion 14c.

By forming the above-described side holes in this manner, a plurality of exhaust routes of the gas G_{L} in the abdominal cavity 72 to the outside of the body are obtained in addition to the route from the distal end portion 14b described above. The applicator 10 of the present embodiment has such a gas leak function for exhausting the gas G_{L} in the abdominal cavity 72 to the outside of the body as described above.

The two side holes 15a at the distal end portion 14b side of the sheath 14 are configured (sized) so that the total area of the two side holes 15a is 6.28 mm², for example in order to assure a leak flow amount of 2 to 4 L/min when the intraperitoneal pressure is 8 to 12 mmHg. Also the two side holes 15b at the rear end portion 14c side of the sheath 14 are configured (sized) so that the total area of the two side holes 15b is 6.28 mm².

If the total area is greater than the noted areas, then the leak flow amount exceeds 4 L/min. In the trocar 50, in order to maintain the intraperitoneal pressure of 8 to 12 mmHz for the leak amount of the gas G_{L} in the abdominal cavity 72, the sterile gas Gv is supplied from the gas supplying unit 60 under the control of the control unit. However, if the leak flow amount exceeds 4 L/min, then the supply amount becomes greater.

On the other hand, if the total area is smaller than the areas described above, then the leak flow amount becomes lower than 2 L/min, and there is the possibility that, at the time of treatment in which the applicator 10 is used, the intraperitoneal pressure may rise exceeding 8 to 12 mmHg.

In the present embodiment, preferably the size of the side holes 15a and 15b is equal to or smaller than 3 mm in diameter where the leak flow amount is taken into consideration. Particularly preferably, the size is 2 mm in diameter.

Further, although the number of side holes at the same formation position (longitudinal or axial position) is two in the present embodiment, the number is not limited to two but may be three or more. If the number of side holes provided at the same formation position (longitudinal or axial position) is one, then the nozzle main body 22 may be one-sided and brought into contact with the inner face 14a of the sheath 14, and thereupon, the side hole may possibly be closed up. In this case, the closed up side hole no more functions as the exhausting route of the gas G_{L} in the abdominal cavity 72. Therefore, the number of side walls at the same formation position is 2 or more.

The formation positions of the side holes in the longitudinal (axial) direction of the sheath 14 and the number of side holes in a circumferential direction of the sheath 14 at each of the same formation positions (longitudinal or axial positions) and so forth are not particularly limited but can be determined suitably if the intraperitoneal pressure can be held at 8 to 12 mmHg and the leak flow amount can be made 2 to 4 L/min.

The sheath 14 is configured from a material which can regulate the shape of the curved portion of the nozzle head 24 when the curved portion of the nozzle head 24 is covered partly or entirely with the sheath 14. An example of the material of the sheath 14 is polyethylene.

On the spray head 20 which forms a part of the nozzle 12, a first connection portion 30a to be connected to a first syringe 34a and a second connection portion 30b to be connected to the second syringe 34b are provided at the opposite side (end) to the side (end) at which the nozzle main body 22 is provided.

A first inner pipe or tube 32a is connected to the first connection portion 30a. The first inner pipe 32a is provided for jetting first liquid, supplied to the first inner pipe 32a from the first syringe 34a, from the nozzle head 24. The first inner pipe 32a is fitted in the nozzle main body 22 and is further connected to the nozzle head 24.

A second inner pipe or tube 32b is connected to the second connection portion 30b. The second inner pipe 32b is provided for jetting second liquid, supplied to the second inner pipe 32b from the second syringe 34b, from the nozzle head 24. The second inner pipe 32b is fitted in the nozzle main body 22 and is further connected to the nozzle head 24.

The first syringe 34a and the second syringe 34b are connected to a pushing unit 36. The pushing unit 36 is provided for pushing the first syringe 34a and the second syringe 34b. The configuration of the pushing unit 36 is not limited specifically and may be of any of the manual operation type and the automatic operation type only if the pushing unit 36 can push the first syringe 34a and the second syringe 34b.

The first syringe 34a and the second syringe 34b are pushed by the pushing unit 36. Consequently, the first liquid can be supplied into the first inner pipe 32a and the second liquid can be supplied into the second inner pipe 32b readily and with certainty. The pushing operation of the pushing unit 36 can be carried out at a desired timing by an operation of the applicator 10 by an operator.

The first liquid filled in the first syringe 34a and the second liquid filled in the second syringe 34b are different in composition from each other.

The first liquid and the second liquid are selected suitably in accordance with an application, an intended usage, a patient and so forth. For example, where the applicator 10 is used for application of anti-adhesion material, for example, one of the first liquid and the second liquid is liquid containing carboxymethyl dextrin, which have been modified with a succinimidyl group while the other is liquid containing sodium carbonate and sodium hydrogen carbonate.

On the other hand, where the applicator 10 is used for application of biological tissue adhesive, one of the first liquid and the second liquid is liquid containing thrombin and the other is liquid containing fibrinogen.

If the first liquid and the second liquid of any of such combinations as described above are mixed, then they gelate. As a result of the gelation, for example, the mixture of the first liquid and the second liquid (hereinafter referred to as “mixed solution”) can stay at the applied biological tissue (target region) with certainty. Further, since the mixed solution stays at the target region with certainty, it can exhibit its function as the biological tissue adhesive or the anti-adhesion material with certainty at the applied biological tissue (target region).

The first and second liquids are not limited to the types and combinations of liquids described above.

A port 29 is provided on the spray head 20 and communicates with the nozzle main body 22. A gas supplying unit 38 is provided for connection to and communication with the port 29 through a tube 37. The port 29 functions as a connection port to a gas supply port of the gas supplying unit 38.

The gas supplying unit 38 includes a gas tank in which sterile gas G is filled at a high pressure. The sterile gas G is provided for jetting mixed solution Lc hereinafter described, and for example, nitrogen gas or the air is used as the sterile gas G.

The sterile gas G can be supplied at a relatively high flow speed to the nozzle head 24 from the gas supplying unit 38. An on-off valve for controlling the sterile gas G between a supply state and a stop state is installed in the gas supplying unit 38 or the tube 37. When the mixed solution Lc hereinafter described is to be applied, the valve is placed into an on state.

The supply unit is configured from, or comprised of, the first syringe 34a and the second syringe 34b as well as the pushing unit 36 and the gas supplying unit 38.

The nozzle main body 22 has a shape of a pipe configured, for example, from stainless steel and is configured from a hollow stainless steel shaft. The nozzle main body 22 has a length of, for example, 30 cm. As described above, the first inner pipe 32a and the second inner pipe 32b are fitted inside the nozzle main body 22, and the sterile gas G passes through the inside of the nozzle main body 22.

The nozzle head 24 is provided at a distal end portion of the nozzle main body 22. The nozzle head 24 is hollow and has a nozzle portion 26 provided in the inside of the nozzle head 24. The first inner pipe 32a and the second inner pipe 32b are connected to the nozzle portion 26, and the first liquid supplied through the first inner pipe 32a and the second liquid supplied through the second inner pipe 32b are mixed with each other in the nozzle portion 26.

The nozzle portion 26 is inserted partly in an opening 24a of the nozzle head 24, and, for example, the other portion of the nozzle portion 26 than the portion inserted in the opening 24a is formed from a porous material.

Consequently, by an operation of the pushing unit 36, the first liquid and the second liquid are supplied to the nozzle portion 26, and the mixed solution Lc in the nozzle portion 26 can be ejected with certainty from the opening 24a by the sterile gas G flowing into the nozzle portion 26 through the inside of the nozzle main body 22 from the gas supplying unit 38. The mixed solution Lc is a mixture of the first liquid, the second liquid and the sterile gas G.

When the nozzle head 24 is jetting the mixed solution Lc, the sterile gas G passing through the nozzle portion 26 becomes microbubbles in the mixed solution which passes through the nozzle portion 26. By virtue of the microbubbles, the mixed solution Lc is agitated in the process of passing through the nozzle portion 26. Consequently, the first liquid and the second liquid are mixed uniformly and with certainty and are injected as the mixed solution Lc from the opening 24a. Especially, when the two liquids are different from each other in viscosity, although uniform mixture solution is less likely to be obtained if the liquids are merely merged, by utilizing the microbubbles, an agitation action of agitating the first liquid and the second liquid to promote mixture of them is manifested. Consequently, the uniform mixed solution Lc is obtained.

The nozzle head 24 has flexibility and is curved such that, for example, the distal end of the nozzle head 24 is directed to an oblique upper side. The axial line g₂ of the nozzle head 24 is inclined by a predetermined angle (other than 0° and 180°) with respect to the axial line g₁ of the nozzle main body 22.

The inclination angle θ of the axial line g₂ of the nozzle head 24 with respect to the axial line g₁ of the nozzle main body 22 when the nozzle head 24 is in a curved state without being regulated by the sheath 14 hereinafter described preferably is approximately 30 to 90 degrees, and more preferably is approximately 70 to 90 degrees.

The curved portion of the nozzle head 24 is configured, for example, from a soft material, an elastic material or the like. Note that a portion of the nozzle head 24 at the proximal end side with respect to the curved portion may be configured from a hard material or else may be configured from a soft material, an elastic material or the like having flexibility.

Further, the nozzle head 24 may be configured such that the curved portion of the nozzle head 24 and the portion of the nozzle head 24 at the proximal end side with respect to the curved portion are configured from separate members and fixed to each other by adhesion, fusion or the like or may be configured otherwise such that the two portions are formed as a unitary member.

The nozzle head 24 may have a configuration disclosed, for example, in the FIGS. 18 to 26 of U.S. Patent Application Publication No. 2009/0124986. Although the nozzle portion 26 is partly configured from a porous material as described above, the nozzle portion 26 is not limited to this and may be entirely configured from a porous material.

The nozzle main body 22 has, for example, two crushed portions 28a and 28b provided at a proximal end portion 28 of the nozzle main body 22 at the spray head 20 side as depicted in FIGS. 3(a) and 3(b).

Each of the crushed portions 28a and 28b is formed by pressing the proximal end portion 28 of the nozzle main body 22 from opposite sides of the nozzle main body 22 by a press to deform the proximal end portion 28 of the nozzle main body 22 in a direction orthogonal to the pressing direction in which the nozzle main body 22 is pressed by the press (the direction orthogonal to the pressing direction is hereinafter referred to as deformation direction) leaving a space, in which the first inner pipe 32a and the second inner pipe 32b can be fitted (positioned), in the inside of the nozzle main body 22. Each of the crushed portions 28a and 28b is a flattened portion formed by deforming the nozzle main body 22 in a deformation direction as described above. Each of the crushed portions 28a, 28b is an axially extending portion at which the nozzle main body 22 is crushed or deformed relative to the portion of the nozzle main body 22 that is axially adjacent the crushed portion. The crushed portions 28a and 28b each provide an axially extending portion of the nozzle main body 22 that is deformed (reduced in outer dimension at one circumferential part and increased in outer dimension at an other circumferential part so that the outer dimension of the one circumferential part is greater than the outer dimension of the other circumferential part). FIGS. 1, 3(a) and 3(b) illustrate that the outer dimension of the crushed portion 28a of the nozzle main body 22 in the deformation direction is less than the outer dimension of the nozzle main body 22 in the same direction in the axially adjacent portion of the nozzle main body 22 (the portion of the nozzle main body 22 to the left of the crushed portion 28a in FIG. 3(b)). The outer dimension of the crushed portion 28a of the nozzle main body 22 in the direction orthogonal to the deformation direction is greater than the outer dimension of the nozzle main body 22 in the same direction in the axially adjacent portion of the nozzle main body 22. Also, the outer dimension of the axially extending crushed portion 28b of the nozzle main body 22 in the deformation direction is less than the outer dimension of the nozzle main body 22 in the same direction in the axially adjacent portion of the nozzle main body 22 (the portion of the nozzle main body 22 to the right of the crushed portion 28b in FIG. 3(b)). The outer dimension of the axially extending crushed portion 28b of the nozzle main body 22 in the direction orthogonal to the deformation direction is greater than the outer dimension of the nozzle main body 22 in the same direction in the axially adjacent portion of the nozzle main body 22. The nozzle main body 22 thus includes enlarged portions (deformed portions) at which the outer dimension of the nozzle body 22 is enlarged relative to (greater than) the axially adjacent portion of the nozzle main body 22. Such enlarged portions contact the inner face of the sheath 14, while the axially adjacent portion of each enlarged portion is spaced from the inner face of the sheath 14. The crushed portions 28a, 28b of the nozzle main body are both positioned closer to the proximal end of the sheath 14 than the distal end of the sheath 14.

The crushed portion 28a and the crushed portion 28b are formed contiguously to each other with the pressing directions with respect to the axial line g₁ of the nozzle main body 22 displaced by a predetermined angle α from each other. The displaced angle α is, for example, 90 degrees. In particular, the deformation directions of the crushed portion 28a and the crushed portion 28b are different from each other, and the angle defined by the deformation angles of the crushed portions 28a and 28b is 90 degrees. The displacement angle α and the angle defined by the deformation directions of the crushed portions 28a and 28b are hereinafter referred to also as installation angle.

The crushed portions 28a and 28b contact the inner face 14a of the sheath 14 (see FIG. 3(a)) to such a degree that sliding resistance is generated, and preferably the crushed portions 28a and 28b have a size (outer dimension) which is greater than the inner diameter of the sheath 14 and with which the crushed portions 28a and 28b can push out the outer face of the sheath 14 to the outer side. In this manner, the crushed portions 28a and 28b contact the inner face 14a of the sheath 14 contiguously to each other in different deformation directions from each other. As can be seen from FIG. 1, the outer surface of the portion of the nozzle main body positioned in front of or distally of the distal-most crushes portion 28a is devoid of a crushed portion(s), and the outer surface of this portion of the nozzle main body 22 is spaced from the inner face of the sheath 14.

Where the crushed portions 28a and 28b are formed or configured as described above, if the sheath 14 and the nozzle main body 22 are moved relative to each other in the longitudinal (axial) direction of the sheath 14 in a state in which the crushed portions 28a and 28b contact the inner face 14a of the sheath 14, then the crushed portions 28a and 28b and the inner face 14a of the sheath 14 slidably move. Thereupon, frictional resistance is generated between the inner face 14a of the sheath 14 and the adjacent crushed portions 28a and 28b, and required sliding resistance can be obtained.

If the crushed portions 28a and 28b are provided otherwise in a spaced relationship from each other, then when the sheath 14 is deformed, the sliding resistance is lower similarly as in the alternative case in which a single crushed portion is provided. Therefore, preferably the crushed portions 28a and 28b are provided contiguously each other.

In the present embodiment, since the crushed portion 28a and the crushed portion 28b of the nozzle main body 22 are contiguous and connected to each other in different directions of the sheath 14, the deformation directions or increased diameter directions of the sheath 14 at the contacting portions are different from each other. Therefore, the sliding resistance between the nozzle main body 22 and the sheath 14 can be enhanced. For example, even if the sheath 14 is deformed or increased in diameter by heating upon sterilization in which an autoclave is used or by time dependent variation or the like of the sheath 14 (i.e., over time the sheath 14 may deform slightly and so contact with the crushed portions 28a, 28b might not be so strong), particularly if the deformation or diameter increase relates to only one of the two directions, then ones of the crushed portions 28a and 28b contiguous to each other can maintain the state in which they contact the inner face 14a of the sheath 14. Therefore, a drop of the sliding resistance can be suppressed. Consequently, degradation of the operability of the applicator 10 by deformation of the sheath 14 can be suppressed. Besides, the possibility that deformation or diameter increase in one direction may occur before deformation or diameter increase in two different directions may occur is high, and therefore, the applicator 10 can cope also with the time dependent variation and so forth of the sheath 14 and can achieve a stabilized operability over a long period of time.

Even if the sheath 14 is deformed by heating upon sterilization in which an autoclave is used, by time dependent variation or the like of the applicator, if a drop of the sliding resistance can be suppressed, then the number of crushed portions is not limited specifically.

As regards the size of the crushed portions 28a and 28b, for example, the length in the longitudinal (axial) direction is 3 to 10 mm, and preferably is 4 to 6 mm. Meanwhile, the width of the crushed portions 28a and 28b in a diametrical direction is, for example, greater by 0.1 to 0.9 mm than the inner diameter of the sheath 14, and preferably is greater by 0.2 to 0.6 mm.

Although the effect of the crushed portions 28a and 28b can be exhibited if the number of the crushed portions 28a and 28b is equal to or greater than two, preferably the number is two. If the number of crushed portions 28a and 28b is excessively great, then there is the possibility that the bending strength of the nozzle may drop.

The installation angle of the crushed portions 28a and 28b preferably is 90±30 degrees (60 to 120 degrees), and more preferably is 90±20 degrees (70 to 110 degrees).

Meanwhile, the contiguous interval (axial or longitudinal distance) between the crushed portions 28a and 28b preferably is 2 to 20 mm, and more preferably is 3 to 10 mm. If the interval between the crushed portions 28a and 28b is smaller, then it is difficult to work the crushed portions 28a and 28b. On the other hand, if the interval between the crushed portions 28a and 28b is excessively great, then a drop of the sliding resistance by the time dependent variation cannot be suppressed.

Meanwhile, if the crushed portions 28a and 28b are provided, for example, at an intermediate region of the nozzle main body 22 in the longitudinal (axial) direction, then the workability is degraded significantly in that the crushed portions 28a and 28b are caught by the opening 56 of the trocar 50 or the like. Therefore, the crushed portions 28a and 28b are provided at the proximal end portion 28 of the nozzle main body 22.

Further, since the crushed portions 28a and 28b have high sliding resistance and the sheath 14 can be moved and stopped and then kept stopped at a predetermined stopping position, the crushed portions 28a and 28b function as positioning means for carrying out positioning of the sheath 14 in the longitudinal (axial) direction of the nozzle 12 at the stopping position. Consequently, the mixed solution Lc can be jetted in a state in which the nozzle head 24 is kept at the predetermined inclination angle θ.

Further, the applicator 10 is used in a state in which it is inserted in the trocar 50 as described above. If, in this state, the applicator 10 is pushed in a direction toward the distal end of the trocar, then outer peripheral portions of the sheath 14 at which the crushed portions 28a and 28b are positioned abut with edge portions of the opening 56 of the main body 52. Therefore, a limit to the movement of the sheath 14 in the direction toward the distal end with respect to the trocar 50 can be regulated. Consequently, the sheath 14 of the applicator 10 can be prevented from inadvertently entering the trocar 50, and the crushed portions 28a and 28b function also as regulation means for regulating the limit to the movement of the sheath 14 in the direction toward the distal end with respect to the trocar 50.

In the present embodiment, the sliding resistance of the sheath 14 by the crushed portion 28a and the crushed portion 28b of the nozzle main body 22 preferably is 3.0 to 11.0 N. The crushed portions are formed with the size, number and installation angle determined such that such sliding resistance as just mentioned can be achieved. The sliding resistance of the sheath 14 was measured in the following manner.

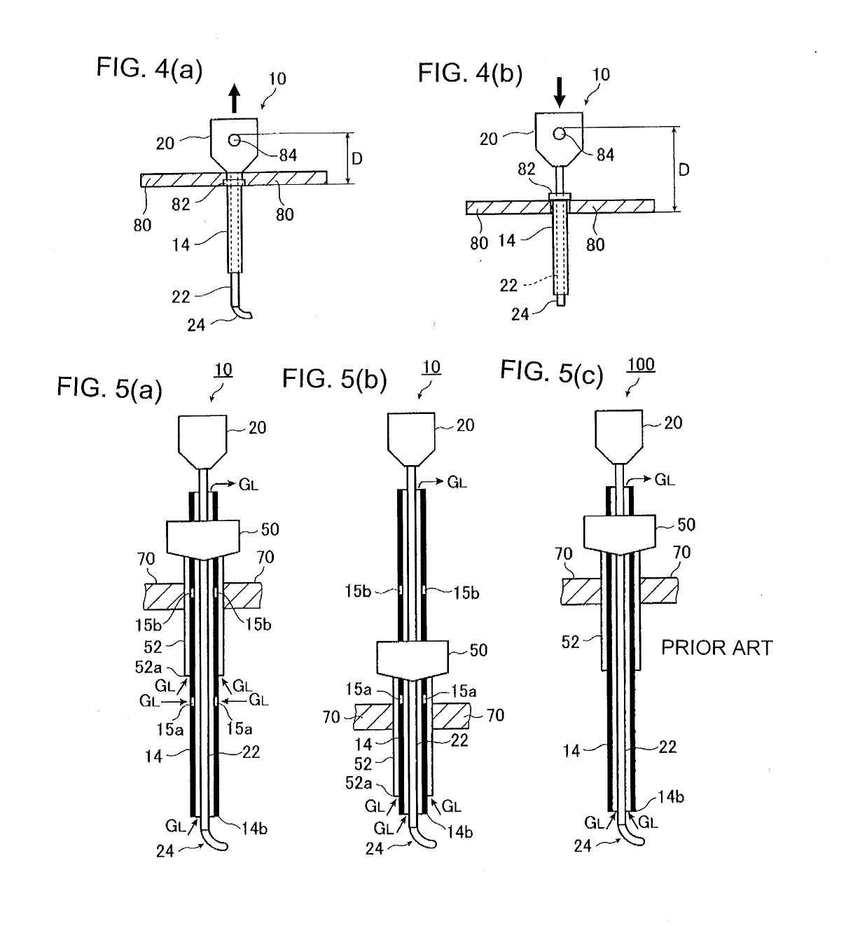

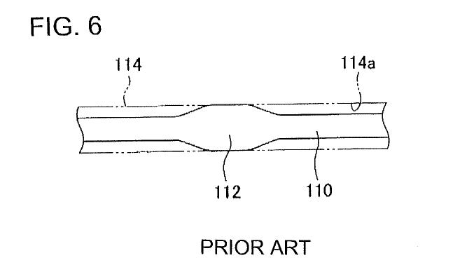

The measuring method of the sliding resistance of the sheath is described with reference to FIGS. 4(a) and 4(b). In FIGS. 4(a) and 4(b), like components to those of the applicator 10 depicted in FIG. 1 are denoted by like reference symbols, and a detailed description of such features is not repeated.

First, the measuring method of the sliding resistance of the sheath when the nozzle main body 22 is pulled is described.

As depicted in FIG. 4(a), a fixing jig 80 was installed on an autograph, and a flaring unit 82 was used to fix the applicator 10 to the fixing jig 80 with the nozzle head 24 positioned on the lower side. Then, the spray head 20 was grasped by the chuck of the autograph on the load cell side.

Then, the spray head 20 was pulled in accordance with tensile test conditions given below, and the maximum force which was generated till a point of time immediately before the nozzle head 24 was brought into contact with the sheath 14 was measured. Then, the maximum force was determined as the sliding resistance value of the sheath 14 when the nozzle main body 22 was pulled.

As the tensile test conditions, the chuck distance D (refer to FIG. 4(a)) was set to 31.5±0.5 mm; the tensile speed was set to 100 mm/minute; and the stroke distance was set to 17.0 mm.

The chuck distance D is a chuck distance from the plane of the fixing jig 80 to a protrusion denoted by reference numeral 84. The protrusion 84 corresponds to the port 29 in FIG. 1 and is a connection portion for the gas supply port.

Now, the measuring method of the sliding resistance of the sheath when the nozzle main body 22 is pushed is described.

As depicted in FIG. 4(b), the fixing jig 80 was placed on the autograph, and the flaring unit 82 was used to fix the applicator 10 to the fixing jig 80 with the nozzle head 24 positioned on the lower side. Then, the spray head 20 was grasped by the chuck of the autograph on the load cell side.

Then, the spray head 20 was pushed in according to the pushing-in conditions given below, and maximum force which was generated till a point of time immediately before the spray head 20 was brought into contact with the sheath 14 was measured. Then, the maximum force was determined as the sliding resistance value of the sheath 14 when the nozzle main body 22 is pushed.

As the pushing-in conditions, the chuck distance D (refer to FIG. 4(b)) was set to 31.5±0.5 mm, and the pushing-in speed was set to 100 mm/minute.

In the applicator 10, when the sheath 14 is moved relative to the nozzle main body 22 along the longitudinal (axial) direction of the nozzle main body 22, the curved portion of the nozzle head 24 is inserted into (enters) the sheath 14. By adjusting the projection length of the curved portion of the nozzle head 24 from the distal end of the sheath 14, the shape of the curved portion can be changed. Consequently, the inclination angle θ of the axial line g₂ of the nozzle head 24 with respect to the axial line g₁ of the nozzle main body 22, namely, the direction of the nozzle head 24, can be adjusted. In particular, for example, the sheath 14 is movable between a first position (inclination angle θ=0 degrees) in which the curved portion of the nozzle head 24 is regulated into a linear shape by the sheath 14 and the direction of the axial line g₂ of the nozzle head 24 and the direction of the axial line g₁ of the nozzle main body 22 coincide with each other, and a second position (the inclination angle θ is the maximum inclination angle) in which the curved portion is in the curved state without being regulated by the sheath 14 and the axial line g₁ of the nozzle main body 22 is inclined with respect to the axial line g₂ of the nozzle head 24. Therefore, by moving the relative position of the sheath 14 and the nozzle main body 22 to a predetermined position between the first position and the second position, the inclination angle θ of the nozzle head 24 can be adjusted freely within the range from 0 degrees to the maximum inclination angle.

In the present embodiment, the crushed portions 28a and 28b exert high sliding resistance with respect to the inner face 14a of the sheath 14 and can stop the sheath 14 at a predetermined stopping position as described above. Besides, the crushed portions 28a and 28b have withstanding property against heating upon sterilization and time dependent variation as described above, and the high sliding resistance with the inner face 14a of the sheath 14 can be maintained over a long period of time. Therefore, the curved portion of the nozzle head 24 can be regulated accurately by the sheath 14, and the mixed solution Lc can be applied accurately keeping the inclination angle θ of the nozzle head 24 at the predetermined angle. Further, even if the applicator 10 is used repetitively, the mixed solution Lc can be applied in such a manner that the inclination angle θ of the nozzle head 24 is kept at the predetermined angle stably and accurately over a long period of time.

In this manner, while the sheath 14 is moved to suitably adjust the inclination angle θ to change the inclination angle θ of the nozzle head 24, the mixed solution Lc can be applied in such a manner as described above from the opening 24a of the nozzle head 24 toward a plurality of locations in the abdominal cavity 72, for example, toward internal organs and the abdominal wall 70 over a wide range readily, with certainty and stably over a long period of time.

In the applicator 10, by suitably setting the degree of the curve (inclination angle θ) of the curved portion of the nozzle head in a natural state in which no external force is applied thereto, for example, if the applicator 10 is formed in a “U” shape, then the mixed solution Lc can be applied also to the abdominal wall 70.

Here, FIGS. 5(a) and 5(b) are schematic views illustrating usage patterns (manner of use or operation) of the applicator according to the embodiment disclosed by way of example, and FIG. 5(c) is a schematic view of a usage pattern of a conventional applicator. In FIGS. 5(a) and 5(b), like elements to those of the applicator 10 depicted in FIG. 1 are denoted by like reference symbols, and a detailed description of such elements is not repeated.

As depicted in FIG. 5(a), when the applicator 10 is inserted toward the abdominal cavity 72 such that the side holes 15b at the upper side (rear end portion) are positioned inwardly of, or on the abdominal cavity side of the outer surface of the abdominal wall, a total of three exhaust routes for the gas G_{L} in the abdominal cavity 72 are available including a first route which passes the distal end portion 14b of the sheath 14, a second route which passes the side holes 15a, and a third route which passes the side holes 15b. Therefore, if the distal end portion 14b of the sheath 14 is immersed in or closed up with liquid like ascites (abdominal cavity fluid), solution used to clean a surgical site (e.g., physiological saline solution) or the like existing in the abdominal cavity 72, then even if the pressure in the abdominal cavity 72 becomes high as a result of injection of the mixed solution Lc for which the sterile gas G of the applicator 10 is used, the gas G_{L} in the abdominal cavity 72 can be exhausted to the outside of the body through the second route and the third route described above.

On the other hand, when the applicator 10 is inserted into the abdominal cavity 72 such that the insertion length of the applicator 10 in the abdominal cavity 72 is relatively short and the side holes 15b at the upper side exist outside the abdominal wall 70, and such that the side holes 15b are not covered by (i.e., are positioned outside of) the main body 52, as depicted in FIG. 5(b), a total of two exhaust routes for the gas G_{L} in the abdominal cavity 72 are available including the first route and the second route described above. Therefore, if the distal end portion 14b of the sheath 14 is closed up by or immersed in liquid like ascites (abdominal cavity fluid), solution used to clean a surgical site (e.g., physiological saline solution) or the like existing in the abdominal cavity 72, then even if the pressure in the abdominal cavity 72 becomes high as a result of injection of the mixed solution Lc for which the sterile gas G of the applicator 10 is used, the gas G_{L} in the abdominal cavity 72 can be exhausted to the outside of the body through the second route described above.

In the conventional applicator 100 having no side hole as depicted in FIG. 5(c), only the first route is available as the exhaust route for the gas G_{L} in the abdominal cavity 72. Therefore, if the distal end portion 14b of the sheath 14 is closed up by or immersed in liquid like ascites (abdominal cavity fluid), solution used to clean a surgical site (e.g., physiological saline solution) or the like existing in the abdominal cavity 72, then even if the pressure in the abdominal cavity 72 becomes high as a result of injection of the mixed solution Lc for which the sterile gas G of the applicator 10 is used, the gas G_{L} in the abdominal cavity 72 cannot be exhausted to the outside of the body.

As described above, with the applicator 10 of the present embodiment, even if the depth of the insertion of the applicator 10 varies, the gas leak function (gas vent) is maintained without being influenced by the variation of the insertion length. Therefore, even if the pressure in the abdominal cavity 72 becomes high as a result of injection of the mixed solution Lc for which the sterile gas G of the applicator 10 is used, the gas G_{L} in the abdominal cavity 72 can be exhausted to outside of the body and the pressure rise in the abdominal cavity 72 can be suppressed. Furthermore, since the applicator 10 includes the plurality of exhaust routes, even if one of the exhaust routes is closed up, the gas leak function is maintained similarly as described above. Consequently, the pressure rise in the abdominal cavity 72 by use of the applicator 10 can be suppressed.

The detailed description above describes an embodiment of an applicator and method representing en example of the applicator and method disclosed here. The invention is not limited, however, to the precise embodiment and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An applicator configured to be positioned in a living body, the applicator comprising:
a sheath possessing a lumen, and open distal and proximal ends communicating with the lumen of the sheath, the sheath also possessing an inner face surrounding the lumen;
a nozzle including an elongated nozzle main body possessing proximal and distal ends, a spray head connectable to different kinds of liquids to be conveyed through the nozzle main body and mixed with one another, and a nozzle head, the spray head being fixed to the proximal end of the nozzle main body so that the nozzle main body and the spray head move together as a unit, the nozzle head being fixed to the distal end of the nozzle main body so that the nozzle main body and the nozzle head move together as a unit, the nozzle head possessing a distal end at which is located an opening through which the liquids mixed together as a mixed solution are ejected together with a gas, the nozzle main body being axially movably positioned in the lumen of the sheath;
the applicator being insertable into a living body to apply the mixed solution to a region in the living body;
the nozzle main body including an axially extending first portion at which the nozzle main body is deformed in a first deformation direction relative to a portion of the nozzle main body axially adjacent the first portion so that an outer circumferential part of the axially extending first portion of the nozzle main body contacts the inner face of the sheath while the portion of the nozzle main body axially adjacent the first portion is spaced from the inner face of the sheath;
the nozzle main body including an axially extending second portion at which the nozzle main body is deformed in a second deformation direction relative to a portion of the nozzle main body axially adjacent the second portion so that an outer circumferential part of the axially extending second portion of the nozzle main body contacts the inner face of the sheath while the portion of the nozzle main body axially adjacent the second portion is spaced from the inner face of the sheath;
the first and second portions of the nozzle body being located axially contiguous to one another and at a proximal end portion of the nozzle main body; and
the first and second deformation directions being different directions.

2. The applicator according to claim 1, wherein the first deformation direction is at an angle of 90±20 degrees to the second deformation direction.

3. The applicator according to claim 2, wherein the first portion of the nozzle main body and the second portion of the nozzle main body include flattened portions of the nozzle main body.

4. The applicator according to claim 2, wherein the first portion of the nozzle main body and the second portion of the nozzle main body are both positioned closer to the proximal end of the sheath than the distal end of the sheath.

5. The applicator according to claim 1, wherein the first portion of the nozzle main body and the second portion of the nozzle main body include flattened portions of the nozzle main body.

6. The applicator according to claim 1, wherein the first portion of the nozzle main body and the second portion of the nozzle main body are both positioned closer to the proximal end of the sheath than the distal end of the sheath.

7. The applicator according to claim 1, wherein the spray head is connectable to the different kinds of liquids by virtue of the spray head being configured to receive syringes respectively containing the different kinds of liquids.

8. The applicator according to claim 1, wherein a gap exists between the inner face of the sheath and an outer surface of the nozzle main body at a position distal of the first and second potions of the nozzle main body.

9. An applicator configured to be positioned in a living body, the applicator comprising:
a sheath possessing lumen, and open distal and proximal ends communicating with the lumen of the sheath, the sheath also possessing an inner face surrounding the lumen;
a nozzle including an elongated nozzle main body possessing an outer surface, a spray head connectable to different kinds of liquids to be conveyed along the nozzle main body and mixed with one another, and a nozzle head, the nozzle main body possessing a distal end and a proximal end, the spray head being fixed to the proximal end of the nozzle main body so that the nozzle main body and the spray head move together as a unit, the nozzle head being fixed to the distal end of the nozzle main body so that the nozzle main body and the nozzle head move together as a unit, the nozzle head possessing a distal end at which is located an opening through which the liquids mixed together as a mixed solution are ejected together with a gas, the nozzle main body being axially movably positioned in the lumen of the sheath;
the applicator being insertable into a living body to apply the mixed solution to a region in the living body;
the nozzle main body including an axially extending first portion and an axially extending second portion which are contiguous to one another at a proximal end portion of the nozzle main body;
one circumferential part of the axially extending first portion possessing an outer dimension in a first direction that is greater than the outer dimension of an other circumferential part of the axially extending first portion, the outer dimension of the one circumferential part of the axially extending first portion of the nozzle main body also being greater than the outer dimension of a portion of the nozzle main body axially adjacent the axially extending first portion;
one circumferential part of the axially extending second portion possessing an outer dimension in a second direction that is greater than the outer dimension of an other circumferential part of the axially extending second portion, the outer dimension of the one circumferential part of the axially extending second portion of the nozzle main body also being greater than the outer dimension of a portion of the nozzle main body axially adjacent the axially extending second portion;
the first direction being a direction that is different from the second direction;
the one circumferential part of the axially extending first and second portions contacting the inner face of the sheath; and
the outer surface of the portion of the nozzle main body axially adjacent the first portion being spaced from the inner face of the sheath, and the outer surface of the portion of the nozzle main body axially adjacent the second portion being spaced from the inner face of the sheath.

10. The applicator according to claim 9, wherein the first direction is at an angle of 90±20 degrees to the second direction.

11. The applicator according to claim 10, wherein the first portion of the nozzle main body and the second portion of the nozzle main body are both positioned closer to the proximal end of the sheath than the distal end of the sheath.

12. The applicator according to claim 9, wherein the first portion of the nozzle main body and the second portion of the nozzle main body are both positioned closer to the proximal end of the sheath than the distal end of the sheath.

13. The applicator according to claim 9, wherein the spray head is connectable to the different kinds of liquids by virtue of the spray head being configured to receive syringes respectively containing the different kinds of liquids.

14. The applicator according to claim 9, wherein a gap exists between the inner face of the sheath and the outer surface of the nozzle main body, the gap extending from the distal end of the sheath to a position at which the first portion of the nozzle main body contacts the inner face of the sheath.

15. An applicator comprising:
a nozzle including an elongated nozzle main body, to which gas and a plurality of kinds of liquids are supplied, and a nozzle head at a distal end of the nozzle main body and configured to jet a mixed solution of the gas and the plurality of kinds of liquids supplied to the nozzle main body;
a sheath in which the nozzle main body is positioned for relative movement along a longitudinal direction of the nozzle main body;
the applicator being insertable into a living body to apply the mixed solution to a region in the living body;
the nozzle main body including at least two crushed portions at each of which the nozzle main body is deformed in a respective deformation direction, the at least two crushed portions being positioned contiguously to each other at a proximal end portion of the nozzle main body that is opposite the nozzle head such that the deformation directions of the two crushed portions with respect to an axial line of the nozzle main body are different from each other; and
the at least two crushed portions individually contacting an inner face of the sheath.

16. The applicator according to claim 15, wherein the different deformation directions of the two crushed portions define an angle of 90±20 degrees between one another.

17. The applicator according to claim 16, wherein the crushed portions are flattened portions of the nozzle main body deformed by being crushed from opposite sides.

18. The applicator according to claim 15, wherein the crushed portions are flattened portions of the nozzle main body deformed by being crushed from opposite sides.

19. The applicator according to claim 15, wherein the nozzle main body possesses a proximal end connected to a spray head, the spray head including first and second connection portions each configured to be connected to a respective syringe containing one of the different kinds of liquids.

20. The applicator according to claim 15, wherein a gap exists between the inner face of the sheath and an outer surface of the nozzle main body at a position distal of all of the crushed portions.
